(19) United States
(12) Patent Application Publication
(10) Pub. No.: US 2009/0076607 A1
(43) Pub. Date: Mar. 19, 2009

(54) Collapsible and Expandable Device and Methods of Using Same

(76) Inventors: Arthur Martinus Aalsma, Zutphen (NL); Hubertus Paul Ter Braak, St. Isidorushoeve (NL)

Correspondence Address: Beusse Wolter Sanks Mora & Maire, 390 N. ORANGE AVENUE, SUITE 2500, ORLANDO, FL, 32801, US

(21) Appl. No.: 11/862,401

(22) Filed: Sep. 27, 2007

(30) Foreign Application Priority Data
Sep. 19, 2007 (GB) 0718200.9

(51) Int. Cl. A61F 2/44

(52) U.S. Cl. 623/17.16; 623/17.11

(57) ABSTRACT

The present invention provides a device (10) suitable for insertion between vertebral portions and having a first, collapsed, position and a second, extended, position, wherein the device comprises upper and lower supports (18, 20) and side supports (22, 24) pivotally connected thereto and in which one of said side supports includes a reaction surface (34) against which, in operation, an actuation member (36) acts so as to cause opening of said device.

This application claims priority to UK application Serial No. 0718200.9 filed Sep. 19, 2007

### FIELD

The present invention relates to an expandable device having first and second support members and having collapsed and expanded positions together with a mechanism for moving said members apart and relates particularly, but not exclusively, to such a device suitable for use in the repair of defective or damaged bone structures such as, for example, vertebral bodies, disk material between said bodies and the interior portions of other bone structures. The invention also relates to methods of using such a device.

### BACKGROUND

It is known that bone tissue inside, for example, a vertebra deteriorates due to illnesses, such as osteoporosis, trauma and the like, and that any surrounding bone tissue may then be subjected to an ever increasing pressure, which may lead to said surrounding tissue collapsing or the vertebra being compressed, with all the unpleasant consequences thereof. In view of this problem there already exist a number of methods of repairing the deterioration and a number of instruments and prostheses for use in the known methods, some of which are discussed below.

WO 2001/03616, for example, discloses a method of restoring the height of a relatively healthy vertebral body in which upper and lower support plates are coupled to each other by articulated side members hinged at their mid position and operable to assist the upper and lower supports move apart in a substantially parallel relationship. Bone material can be inserted into the space defined by the support so as to further strengthen the supporting structure and restore bone properties.

WO 1998/56301 discloses a method in which the height of a crushed vertebra is restored by inserting an inflatable balloon into the cavity within the vertebra. The balloon is first inserted in a deflated state through a small opening in the vertebral wall and into said vertebral cavity after which it is inflated, as a result of which the vertebra regains its original condition. Then the balloon is deflated and removed, after the space created inside the vertebra is filled with bone cement which, when hardened, restores the mechanical strength of the treated bone. This technique is also described in relation to hip and other joints. One drawback of this method resides in the fact that the inserted material is subjected to a pressure upon deflation of the balloon; as a result of which said material may leak out, so that it will no longer perform its function to its full extent. Furthermore, the quality of the fusion between the inserted material and the surrounding bone tissue may not be fully satisfactory, resulting in a less than optimum long-term strength and quality of the treated vertebra.

WO2003/003951 discloses an instrument for insertion into the vertebral body and includes upper and lower support portions for supporting the vertebra and a mechanism for expanding same such as to restore the vertebral body. The mechanism itself comprises a somewhat complex arrangement of a sliding beam shaped element slidable along a lower surface of the upper support and a pair of parallel side arms which are pivotally arranged relative to the beam element and the lower support such as to allow expansion and contraction of said instrument upon activation of an activation mechanism engageable with one of said parallel side arms. Whilst this arrangement provides a perfectly acceptable mechanism for use in certain applications, the structure thereof is somewhat complex and does not lend itself to use in confined situations.

### SUMMARY

It is an object of the present invention to provide a device for expanding the vertebral body that may be used as an instrument that is removed after bone repair material has been inserted or that may be retained therein as an implant which reduces and possibly eliminates the disadvantages associated with the above-mentioned devices.

Accordingly, the present invention provides a device for insertion between vertebral portions and having a first collapsed position and a second extended position which comprises: a first (upper) support member; a second (lower) support member; and first and second side supports; wherein, said side supports are pivotally connected to said upper and lower members and wherein one or more of said side supports includes a reaction surface against which, in operation, an actuation member reacts to cause opening of said instrument from said collapsed to said extended position.

In one arrangement the side supports extend parallel to each other so as to provide a particularly rigid support structure. However, other arrangements are possible as discussed herein.

For convenience of packaging and insertion, the first support member may comprise two or more articulated portions, each of which is hinged relative to its neighbor.

Preferably, said second support member comprises two or more articulated portions.

Advantageously, the combined pivoted length of said first (upper) support and said first side support is substantially equal to the combined pivoted length of said second (lower) support and said second side support.

Preferably, said reaction surface comprises a cam surface against which an actuation member may react upon axial displacement of said reaction member, thereby to cause said side support to pivot about its pivotal connection point and move between a collapsed and an extended position.

Advantageously, said side support includes a second cam surface against which an actuation member may react upon axial displacement of said reaction member, thereby to cause said side support to pivot about its pivotal connection point and move between an extended and a closed position. The first cam may actually comprises two cam surfaces so as to ensure even load distribution, as may the second cam surface. Indeed, the first and second cam surfaces may be defined by a slot within a side portion of said side support.

Preferably, said first (upper) support member further includes an extension portion adjacent a pivot point with an associated side support and said extension portion is connectable to an actuation mechanism. The extension portion may be pivotally connected to the extension portion. Such an arrangement allows the supports to pivot relative to the extension portion and will assist with the placement and actuation of the device itself.

Advantageously, the device includes a lock mechanism for locking said instrument in a position between fully collapsed and fully extended positions.

In one possible arrangement said lock mechanism comprises one or more recesses within one or more of said cam surfaces and into which said reaction member may be lockably located.

The device described so far may be used on its own as in implant or may include the addition of an actuation member so as to form an instrument for manipulation by a surgeon. Such an actuation member may comprise an axially translatable member having a surface for engagement with said cam or cams.

Advantageously, said actuation member includes a carrier portion for carrying said axially translatable member and further includes a locking mechanism for locking said axially translatable member relative to said carrier portion.

In a particularly advantageous arrangement the device further including a separable coupling between said actuation member and said supports, thereby to allow the actuation member to be used to install the device whilst allowing it to be left in its installed position by decoupling the two portions.

In order to assist the operator establish what sort of load he is exerting on the active portion of the device said actuation member further includes a load sensor for sensing the load exerted on the supports and may further include a load display.

The device may further include a mechanically leveraged trigger mechanism for causing axial translation of an actuation mechanism which may comprise a hand operable actuation mechanism and a flexible connection between said hand operable mechanism and said supports.

It will be appreciated that said device may be an implant or prosthesis.

According to another aspect of the present invention there is provided a method for emplacement of a spacer device comprising the steps of: providing a device as claimed in any one of claims 1 to 14; providing an actuation member as claimed in any one of claims 15 to 17; connecting said actuation mechanism to said device for actuation thereof; inserting said device in a collapsed state into a structure to be restored; and causing said device to expand within said structure, such as to cause the support members to engage with sound portions thereof, thereby to cause said portions to be moved apart and to a desired distance from each other to restore or at least partially restore the structure.

The method may include the further step of inserting a bone repair material within a cavity formed by said device.

When the device is an instrument, the method may include the further step of removing said device from said cavity.

Alternatively, the method may include the step of disconnecting said actuation member from said device and withdrawing said actuation member, thereby to leave said device within said structure as an implant or prosthesis.

An advantageous aspect of the invention resides in the fact that the instrument has been designed to be slender and particularly compact whilst being structurally robust such that the surgeon can perform the operation with minimal invasive surgery whilst also ensuring good support either during or after the operation.

Another advantage is that the method employing the instrument according to the present invention is quite similar to generally accepted techniques for restoring inter-vertebral discs, in particular to the so-called back approach. According to said approach, two blocks, also referred to as cages, are inserted into the inter-vertebral disc space on either side of the spinal cord to restore the spacing between the two adjacent vertebrae before fixating the two vertebrae relative to each other. As is the case with the aforesaid conventional techniques for restoring inter-vertebral discs, the present instrument has two different functions, viz. restoration of the vertebra to its normal dimension and bearing the load on the vertebral body until sufficiently bone tissue has formed around the instrument that takes over the load. The present invention also allows the surgeon to insert various kinds of material around the instrument, for example bone cement, bone particles, minerals, etc. in order to accelerate bone growth without adversely affecting the bearing capacity of the instrument.

A still further advantageous aspect is the fact that the restoration of the shape and the dimensions of the vertebral body can easily be checked by the surgeon, with the surgeon being able to withdraw the instrument if he is not absolutely certain that the instrument is correctly positioned.

### BRIEF DESCRIPTION OF THE DRAWINGS

The invention will now be more particularly described by way of example only with reference to the following drawings, in which:

FIG. 1, is a general view of a device according to the present invention when assembled with an actuation mechanism according to another aspect of the present invention so as to form an instrument for use as will be described;

FIG. 2, is an isometric view of the device portion of the present invention;

FIG. 3 is a view in the direction of arrow A in FIGS. 2 and 4,

FIGS. 4 to 7, illustrate collapsed, partially expanded and fully expanded positions of the device;

FIG. 8, is an isometric projection of the instrument in its collapsed state;

FIG. 9, is a detailed cross-sectional view of an actuation mechanism suitable for use with the device of FIGS. 1 to 6;

FIGS. 10 to 12 illustrate the tool expanding within a vertebrae;

FIGS. 13 to 15 illustrate different dimensional arrangements for the present invention; and

FIG. 16 illustrates a number of possible approach angles that a surgeon may adopt when tackling a collapsed vertebra.

### DETAILED DESCRIPTION

Referring now to the drawings in general but particularly to FIG. 1, an instrument 10 includes a device portion 12 and an actuation portion 14, each of which are shown in more detail in subsequent drawings. The device 12 portion and actuator portion 14 may be separated from each other by an actuation shaft 16 which may be of a rigid or a flexible type, again as detailed later herein.

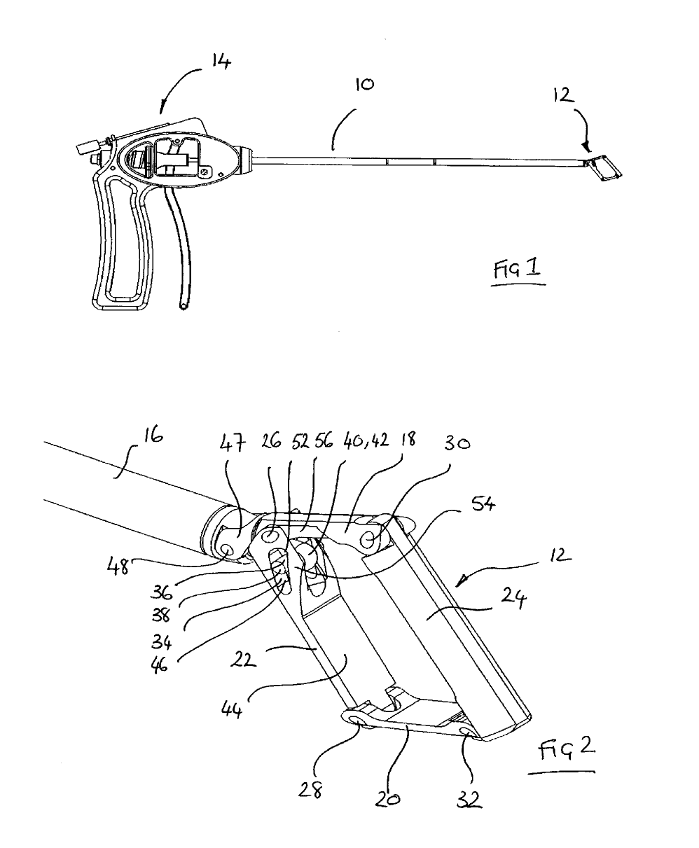

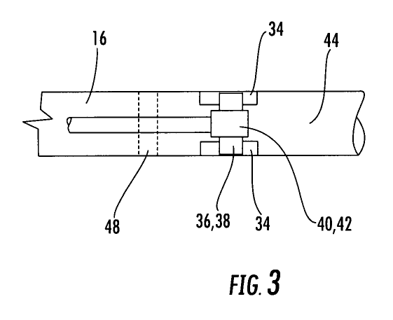

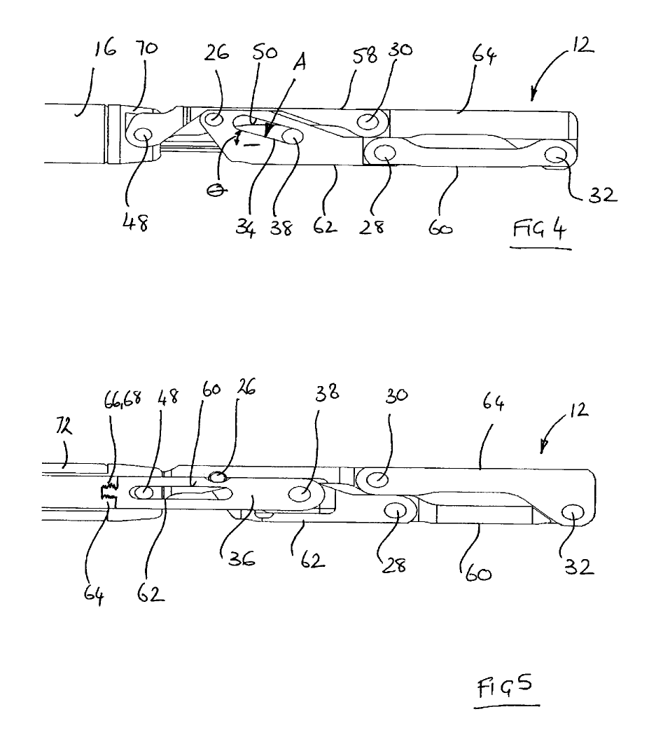

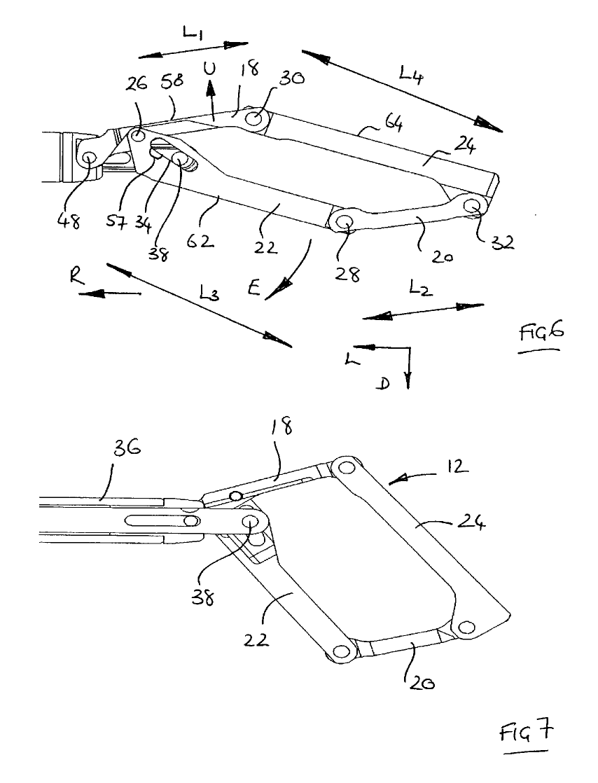

FIG. 2 illustrates the device in more detail and shows it in a partially expanded state in which upper and lower support members 18, 20 are spaced from each other by load carrying side supports 22, 24 each of which are pivotally connected to said upper and lower support members 18, 20 by means of respective pivots 26, 28, 30, 32, which may be pins or any other suitable pivot arrangement. One or other of the side supports 22, 24 is provided with a reaction surface 34 against which, in operation, an actuation member, a portion of which is shown at 36, may operate. Whilst the actuation member 36 is shown more clearly in later drawings, it will be appreciated that it may include a pin arrangement 38 extending to one or more sides of a central actuation rod 40 which may extend along the actuation shaft 16 to the actuator portion 14 of FIG. 1. The rod itself may comprise a rigid rod or a flexible multi-stranded cable. Either arrangement may be provided with an enlarged head portion 42 through which said pin 38 extends such that it engages with the reaction surface 34. The reaction surface 34 may comprise the inner surface 44 of the appropriate side support 22 or may comprise a cam surface 46 specifically provided thereon, said cam surface 46 being angled upwardly at an angle Θ (FIG. 3) relative to the inner surface 44 so as to assist with deployment as will be discussed later. The first or upper support member 18 includes an extension portion 47 which extends said portion laterally from the upper pivot point 26 towards the actuation shaft 16 and which is provided with a further pivot at 48 which pivotally connects said first (upper) support member to said actuation shaft in a manner that will be appreciated in detail from FIGS. 4 to 8. Further features that may be appreciated from FIG. 2 include the provision of cam, surfaces 46 on either side of the actuation rod and the double headed nature of the pin arrangement, which is seen in more detail in FIG. 3. As shown, the device includes optional second cam surfaces 50 provided opposite said first surfaces and against which, in operation, said actuation pins 38 may act so as to contract the assembly. Still further, it will be appreciated that the cam surfaces 46, 50 may be provided as a slot 52 within a raised portion 54 of the side support 18 and that the actuation rod 40 and head portion 42 may pass therebetween in order to correctly position the pin arrangement 38 with respect to said cam surface or surfaces 46. A corresponding portion on the upper support member 18 is wasted or cut away at 56 so as to allow for the assembly to lie one portion within the other, as is shown in the illustration of the collapsed arrangement of FIGS. 4 and 5. Additionally, one or other of the cam surfaces may be provided with a cut-out 57 shaped and dimensioned to correspond with the pin 38 such that said pin can be retained therein by way of an interference fit so as to lock the pin and device in an expanded condition. Finally, the edges of each of the upper and lower supports 18, 20 and side supports 22, 24 may be rounded off as shown by the curved profile of the figures in general, thereby to provide an arrangement in which the cross-sectional profile of the collapsed device is generally circular save for flattened upper and lower support surfaces 58, 60 and side surfaces 62, 64, best seen in FIGS. 4 to 7.

Referring now to FIGS. 4 to 7, which illustrates the device portion extending between a collapsed arrangement and a fully deployed arrangement, it will be appreciated that the profile in the collapsed arrangement is generally slim, with the portions nestling generally one on top of each other with the side supports 22, 24 being positioned in confronting relationship to the upper or lower supports 18, 20. FIG. 3 also aptly illustrates the way in which the wasted portion 58 in the upper support 18 is employed to accommodate the raised side portions 56 which house the cam surfaces 34, 50. The reader will appreciate that pin 38 is positioned at the right hand side of slot 52 and, as long as the pin remains in this position, the device will remain in its collapsed state in which it may be inserted into a vertebral cavity, as will be discussed later herein.

FIG. 5 is a cross-sectional view of the arrangement of FIG. 4 and illustrates in more detail the positioning of the actuation pin 38 within the collapsed side and upper and lower supports. It will be appreciated that in order to accommodate pin 26 it may be necessary to cut a corresponding slot 60 in the actuation member 36 and that said actuation member 36 may be provided with an internal slot 62 for accommodating pivot pin 48 discussed in detail above with reference to FIG. 2. A separable coupling 64 may be provided in the form of corresponding and co-operating threads 66, 68 provided towards the end of actuation member 36 so as to allow a head portion 70 and the device portion 12 to be separated from the actuation mechanism 14, as and when required. In operation, the surgeon need simply twist the rod 40 of the actuation mechanism against the action of the screw thread to cause said rod to disengage from the head portion 70 and allow it to be decoupled, as will be discussed later. The outer sleeve 72 of the actuation shaft is not coupled to the head and is simply held in position by virtue of the screw thread arrangement 66, 68 and its abutment up against the radially extending surface 74 of head portion 70.

Referring now briefly to FIG. 6, from which it will be appreciated that the device portion 12 may be extended by retracting the actuation rod 36 in the direction of arrow R such as to cause pins 38 to react against cam surfaces 34 and cause the side support 22 to pivot about pin 26 and move in the direction of arrow E to a partially extended position, as shown. The actuation of rod 36 also causes the upper support surface 18 to pivot slightly about pivot pin 48 and move upwardly in the direction of arrow U. Indeed, each of the portions 18, 20, 22, 24 will pivot about their respective pivot pins 26. 28. 30, 32 as actuation rod 36 is moved laterally and this results in the lower support 20 moving generally downwardly in the direction of arrow D and laterally in the direction of arrow L, whilst the upper support 18 simply pivots about pin 48 and moves slightly upwards. It will also be appreciated that most of the motion is experienced by the lower support 20 and the side supports 22, 24 and this heralds an advantage which will be explained later herein.

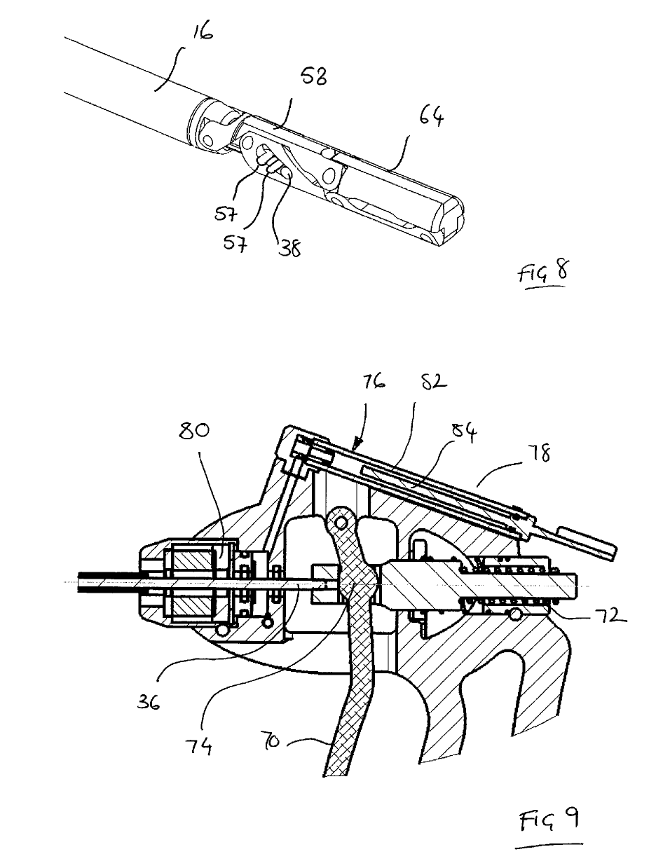

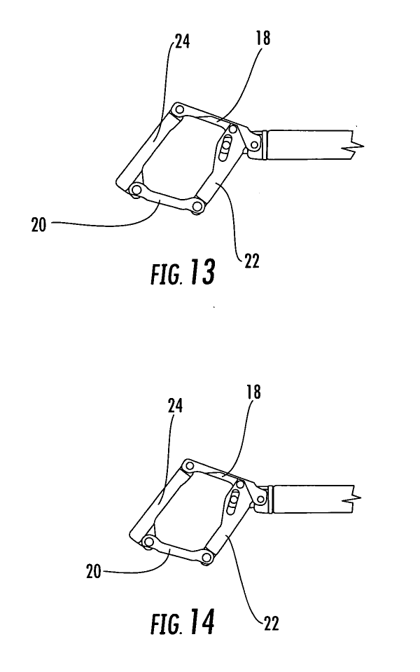

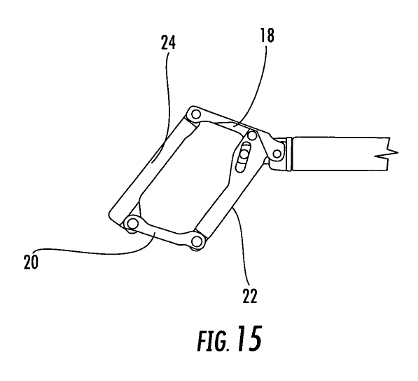

FIG. 7 illustrates the fully expanded position of the supports, from which it will be seen that the side supports 22, 24 are substantially parallel to each other whilst the upper and lower supports 18, 20 are angled relative to each other by an amount determined by the operable length L1 to L4 between the pivot pins 26, 28, 30, 32. In practice, if the operable length L1 is equal to L2 and operable length L3 equal to L4 the opened device 12 will have parallel upper and lower supports 18, 20 and parallel side supports 22, 24. However, if one alters the various lengths it is possible to alter the angular relationship between the upper and the lower supports 18, 20 such as to provide a slope or incline to the device, which feature may be exploited when attempting to restore a prior or desired angular relationship between affected vertebra. Indeed, this feature in combination with the tilting angle induced by pivoting the upper support about pivot pin 48 endow the present invention with advantages over the art which can assist with the correct positioning of the device and provide improved support for the affected bone portions, both during separation and thereafter. Examples of the various length ratios of the supports 18, 20 and 22, 24 are given in FIGS. 13 to 15, from which it will be appreciated that the critical dimensions are measured between the pivot points or hinge points of each support member relative to the next member. The various lengths can be altered to different finished heights and different finished angles of divergence between the upper and the lower supports 18, 20. In the examples the variation in height is between 13.2 mm of FIGS. 13 and 14 and 17.4 mm of FIG. 15. The angle of divergence is shown to vary between 4.8 degrees and 4.9 degrees, although other angles are possible. This angle is selected to suit the angle of natural alignment of the vertebrae between which the instrument is to be inserted and may change as and when necessary.

FIG. 8 illustrates the device in its collapsed state and illustrates the generally circular profile which allows for the easy introduction thereof down a biopsy needle or the like and also illustrates multiple cut-outs 57 for providing multiple locking positions at different heights.

Turning now to FIGS. 1 and 9 which illustrate one possible actuation mechanism 14, it will be appreciated that the function of this device is to displace the actuation shaft 36 laterally as and when desired in order to cause the pins 38 to react against one or other of cam surfaces 34, 50 so as to open or close the device itself as illustrated in FIGS. 4 to 7. To this end, the actuation mechanism may comprise a simple “push-pull” system such as a loop in the end of the actuation shaft 36 which a surgeon, or assistant, may push, or pull, in order to operate the mechanism 14. Alternatively, a more complex and easily controlled arrangement, such as that shown in FIG. 9, may be provided. The arrangement of FIG. 7 includes a pivotally mounted trigger 70 spring biased by spring 72 and coupled at 74 to the actuation rod 36 such that pulling on the trigger 70 causes the actuation rod 36 to be displaced laterally together with pin 38 so as to cause the opening of the device 12, as described above. Additional features may include a load cell or load detector, shown generally at 76, for determining the amount of operator force being exerted on the device and a visual indicator shown generally at 78. Whilst there exist a number of suitable load cells or detectors and visual indicators that may be used, it has been found that a simple hydraulic chamber 80 and pressure tube 82 having a floating indicator 84 within the tube 82, as shown, may be employed in order to give the operator an indication of the pressure being exerted. The pressure exerted being a direct indication of the separation pressure being exerted on the bone structure concerned. The tube 82 may be marked or calibrated to give a visual indication of the pressure being exerted and may still further be marked with a simple traffic light colouring arrangement with Red indicating excessive pressure, Amber indicating acceptable pressure and Green indicating under loading. Alternatives such as strain gauges and the like may be employed.

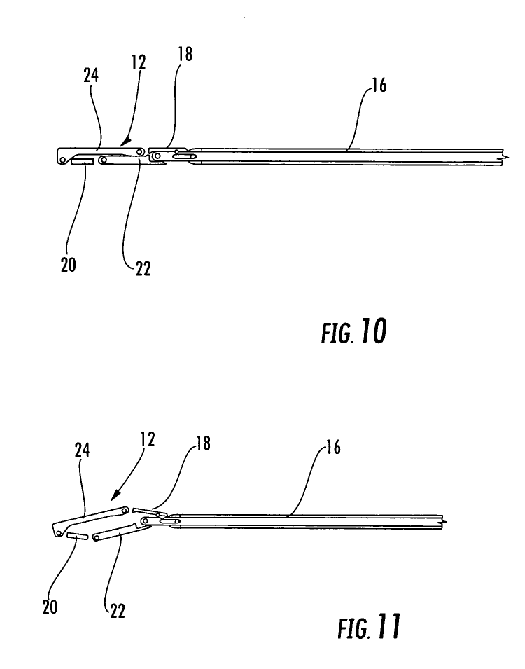

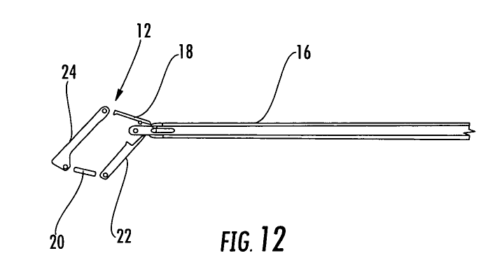

Operation of the above-described arrangement will now be described with reference to FIGS. 10 to 12 and 16, which illustrate the arrangement within a damaged vertebra. In the example shown, the approach is through a small hole previously drilled in the pedicle region and into which a tubular needle (not shown) of internal diameter sufficient to accommodate the present invention is inserted to act as a guide. The device portion is then inserted through the tubular needle such that it emerges from a free end thereof and is positioned between upper and lower damaged or collapsed bone portions 90, 92. A portion of the actuation shaft 16 will remain within the needle which continues to act as both a guide and a support during any subsequent operation. Manipulation of the actuation portion 14 by pulling handle 70 will cause device 12 to expand, as shown progressively in FIGS. 10 to 12. This expansion causes the lower support 20 to move downwardly and backwardly, as described above, which causes the lower bone structure to be progressively pushed downwardly, compressing calcinated material. This movement also pushes the upper and lower portions of the vertebra apart so as open up an inter-vertebral gap, as shown more clearly in FIG. 12. It will be appreciated that whilst the expansion is taking place, the side support 22 will be pivoting about pivot point 26 such that the side support moves downwardly and backwardly in the direction of arrow R in FIG. 6. This action assists with the compaction of any calcinated bone material that might be present in the vicinity and assists with the creation of a stronger and more dense surrounding structure. Whilst this compression technique is of particular benefit in the restoration of vertebrae, it may also be used on a number of other surgical where compaction of diseased or wasted bone material is desired. Some of these techniques are described briefly later herein.

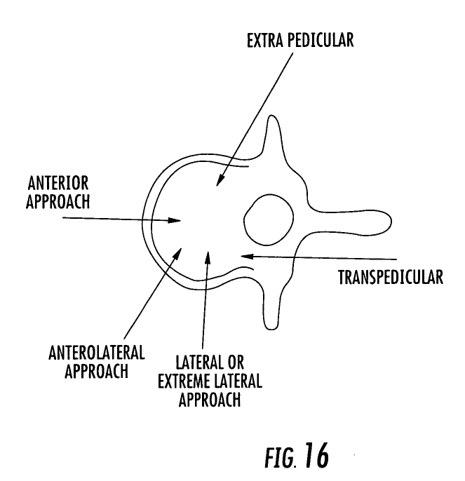

FIG. 16 illustrates the various angular approaches that can be employed with the present device, some of which may not be possible with the arrangements of the prior art.

It will be appreciated that the angular relationship or taper between the upper and lower support surfaces can be altered by altering the lengths L1 to L4, as discussed above. Consequently, if one wishes to provide a support having an angular relationship between these two surfaces one simply need alter the lengths L1 to L4 accordingly. Such a feature is of particular benefit when attempting to restore the structure of a vertebra as it may be used to assist with the recreation of the original relationship between the upper and lower portions of the vertebra rather than simply create a parallel association.

Once the device 10 has been fully expanded the supports 22, 24 act to maintain the distance between separated bone portions whilst also acting to take or share any load passed therebetween. Consequently, the device may be decupled from the actuation portion by disengaging screw threads 66. 68, as described above, and withdrawing the actuation shaft 16 such that the device itself becomes a support implant or prosthesis around which the surgeon may insert optional bone repair material. Alternatively, the surgeon may insert such material before withdrawing the device and allowing the inserted material to take any load.

The above device also lends itself to use in the restoration of other bone structures such as, for example, the inter-vertebral gap in which spinal disk material is present. Indeed, the device may be used to separate the vertebrae on either side of an affected disk such as to allow the disk to be removed, repaired, manipulated or replaced before being withdrawn so as to restore the spine to it's pre-damaged state. Still further, the expansion of the device 10 may be employed in the compacting of calcinated bone material within a bone cavity, such as may be present in a femour of a patient suffering from osteoporosis. In such an arrangement, the device 10 may be inserted into the cavity and repeatedly expanded and contracted as it is withdrawn along the cavity, such as to cause compaction of the inner bone material towards the outer portions of the bone itself. Rotation of the device 10 as it is being withdrawn will further assist with the compaction of material. This compaction process may then be followed by the injection of bone repairing material into the created cavity such as to assist with the creation of a stronger bone structure.

It will be appreciated that whilst the above device has been described with reference to an actuation mechanism working against a side support, such a support may form an upper or a lower support depending on the angle of use.

1. A device for insertion between vertebral portions and having a first collapsed position and a second extended position comprises:
a) a first (upper) support member;
b) a second (lower) support member; and
c) first and second side supports;
wherein, said side supports are pivotally connected to said upper and lower members and wherein one or more of said side supports includes a reaction surface against which, in operation, an actuation member reacts to cause opening of said instrument from said collapsed to said extended position.

2. A device as claimed in claim 1, wherein said side supports extend parallel to each other.

3. A device as claimed in claim 1, wherein said first support member comprises two or more articulated portions.

4. A device as claimed in claim 1, wherein said second support member comprises two or more articulated portions.

5. A device as claimed in claim 1, wherein the combined pivoted length of said first (upper) support and said first side support is substantially equal to the combined pivoted length of said second (lower) support and said second side support.

6. A device as claimed in claim 1, wherein said reaction surface comprises a first cam surface against which an actuation member may react upon axial displacement of said reaction member, thereby to cause said side support to pivot about its pivotal connection point and move between a collapsed and an extended position.

7. A device as claimed in claim 5 wherein said side support includes a second cam surface against which an actuation member may react upon axial displacement of said reaction member, thereby to cause said side support to pivot about its pivotal connection point and move between an extended and a closed position.

8. A device as claimed in claim 6, wherein said first cam comprises two cam surfaces.

9. A device as claimed in claim 7, wherein said second cam comprises two cam surfaces.

10. A device as claimed in claim 9, wherein said first and second cams are defined by a slot within a side portion of said side support.

11. A device as claimed in claim 1, wherein said first (upper) support member further includes an extension portion adjacent a pivot point with an associated side support and said extension portion is connectable to an actuation mechanism.

12. A device as claimed in claim 11, wherein said extension portion is connected to said actuation mechanism by a pivotal connection.

13. A device as claimed in claim 1 including a lock mechanism for locking said instrument in a position between fully collapsed and fully extended positions.

14. A device as claimed in claim 13, wherein said lock mechanism comprises one or more recesses within one or more of said cam surfaces and into which said reaction member may be lockably located.

15. A device as claimed in claim 1 including an actuation member.

16. A device as claimed in claim 15 wherein said actuation member comprises an axially translatable member having a surface for engagement with said cam or cams.

17. A device as claimed in claim 16 wherein said actuation member includes a carrier portion for carrying said axially translatable member and further includes a locking mechanism for locking said axially translatable member relative to said carrier portion.

18. A device as claimed in claim 15 and further including a separable coupling between said actuation member and said supports.

19. A device as claimed in claim 15, wherein said actuation member further includes a load sensor for sensing the load exerted on the supports.

20. A device as claimed in claim 19 and further including load display.

21. A device as claimed in claim 15 and including a mechanically leveraged trigger acutation mechanism for causing axial translation of an actuation member.

22. A device as claimed in claim 15 wherein said actuation mechanism includes a hand operable mechanism and a flexible connection between said hand operable mechanism and said supports.

23. A device according to claim 1 wherein said device is an implant or prosthesis.

24. A method for emplacement of a spacer device comprising the steps of:
providing a device for insertion between vertebral portions and having a first collapsed position and a second extended position, said device comprising a) a first (upper) support member; b) a second (lower) support member; and c) first and second side supports; wherein, said side supports are pivotally connected to said upper and lower members and wherein one or more of said side supports includes a reaction surface against which, in operation, an actuation member reacts to cause opening of said instrument from said collapsed to said extended position: and wherein said reaction surface comprises a cam surface against which an actuation member may react upon axial displacement of said reaction member, thereby to cause said side support to pivot about its pivotal connection point and move between a collapsed and an extended position:
providing an actuation member, said actuation member comprising an axially translatable member having a surface for engagement with said cam or cams;
providing a mechanically leveraged acutation mechanism for causing axial translation of an actuation member;
connecting said actuation mechanism to said device for actuation thereof;
inserting said device in a collapsed state into a structure to be restored; and
causing said device to expand within said structure, such as to cause the support members to engage with sound portions thereof, thereby to cause said portions to be moved apart and to a desired distance from each other.

25. A method as claimed in claim 24 including the further step of inserting a bone repair material within a cavity formed by said device.

26. A method as claimed in claim 25 including the further step of removing said device from said cavity.

27. A method as claimed in claim 25 including the step of disconnecting said actuation member from said device and withdrawing said actuation member, thereby to leave said device within said structure as an implant or prosthesis.

28. A method of compressing tissue of a subject in need thereof, said method comprising:
inserting a device into a site, said device having a first collapsed position and a second extended position, and comprising a) a first (upper) support member, b) a second (lower) support member; and c) first and second side supports; wherein, said side supports are pivotally connected to said upper and lower members and wherein one or more of said side supports includes a reaction surface against which, in operation, an actuation member reacts to cause opening of said instrument from said collapsed to said extended position; and
actuating said device so as to cause opening of said device; thereby exerting compressive forces to tissue proximate to said site.

29. The method of claim 28, further comprising creating a space into which said device is inserted.

30. The method of claim 28 wherein said site is located between two vertebrae.

31. The method of claim 29, wherein said space is created into a bone of said subject.

32. The method of claim 31, wherein said bone is a femur.

33. The method of claim 28, wherein said actuating member is coupled to an actuating mechanism and further comprising decoupling said actuating mechanism from said device upon said actuating step.

34. The method of claim 28, wherein said compressive forces are directed to cause a compaction of calcinated bone.

35. A method of expanding an intervertebral space, said method comprising:
inserting a device into said intervertebral space, said device having a first collapsed position and a second extended position, and comprising a) a first (upper) support member, b) a second (lower) support member; and c) first and second side supports; wherein, said side supports are pivotally connected to said upper and lower members and wherein one or more of said side supports includes a reaction surface against which, in operation, an actuation member reacts to cause opening of said instrument from said collapsed to said extended position; and
actuating said device so as to cause opening of said device; thereby resulting in expansion of said intervertebral space.
